(19) United States
(12) Patent Application Publication
(10) Pub. No.: US 2007/0213780 A1
(43) Pub. Date: Sep. 13, 2007

(54) IMPLANTABLE PULSE GENERATOR

(76) Inventors: Ronald REBENTISCH, Berlin (DE); Roland Jacobsen, Berlin (DE)

Correspondence Address: DALINA LAW GROUP, P.C., 7910 IVANHOE AVE. #325, LA JOLLA, CA, 92037, US

(21) Appl. No.: 11/670,832

(22) Filed: Feb. 2, 2007

(30) Foreign Application Priority Data
Mar. 9, 2006 (DE) 10 2006 010 851.5

(51) Int. Cl. A61N 1/375

(52) U.S. Cl. 607/36

(57) ABSTRACT

To produce implantable devices such as cardiac pacemakers, it is suggested that the housing be assembled from prefinished plastic parts (A, B). Such housing parts may be produced cost-effectively and are very simple to connect to one another using known joining technologies. To achieve the required seal, such a housing may be coated using a special plastic after the assembly, for example. In order to fulfill the requirements for electromagnetic compatibility, interiors of the plastic parts are vapor-deposited using metal. Metal surfaces (3) may be embedded in parts of the housing wall as reference electrodes.

This application takes priority from German Patent Application DE 10 2006 010 851.5 filed 9 Mar. 2006, the specification of which is hereby incorporated herein by reference.

### BACKGROUND OF THE INVENTION

1. Field of the Invention

The present invention relates to implantable devices. In particular, the present invention relates to implantable pulse generators, such as cardiac pacemakers, defibrillators, or cardioverters.

2. Description of the Related Art

In a typical embodiment, an implantable pulse generator comprises various electronic circuits required for generating specific electrical pulses and a long-lived electrical power source for powering these electronics. All components are housed in a sealed housing, the housing being manufactured from metal, generally titanium. The housing thus has a shielding effect in relation to electrical interference fields, so that the electromagnetic compatibility (EMC) is ensured, in addition, the surface of the housing in the body interior may also be used as an electrode for delivered electrical pulses, e.g., as a reference electrode of a cardiac pacemaker. Furthermore, a connection part (header) is fixed on the housing, which comprises epoxide resin and has standardized plug sockets for connecting external electrodes, for example. For contacting, the various electrode lines are guided from and/or to the circuits into this connection part using bushing pins from the housing interior.

In order to manufacture an implantable device of this construction, the housing is typically assembled from two titanium half shells. These half shells are then connected to one another so they are gas-tight and liquid-tight. For this purpose, the half shells are first fixed to one another in an exact position using a laser stitching process, for example, and the peripheral seam is subsequently welded closed in a further manufacturing step. The attachment of the header made of epoxide resin having the various electrical bushings to the housing interior is especially critical. High requirements on the seals are to be set here.

The production of an implantable device in the embodiment described is relatively costly. A significant component of the manufacturing costs arise from the housing: the manufacturing of precisely fitted titanium half shells is complex and accordingly expensive. In addition, the danger always exists when assembling and welding the seam that the seal will not be provided 100%. This is also true for the mounting of the epoxide header. Very expensive bushings are necessary here for leading out gas-tight and liquid-tight electrical connections from the housing.

### BRIEF SUMMARY OF THE INVENTION

The present invention is directed to the described prior art for producing implantable devices. The object of the present invention is to specify an alternative, cost-effective housing embodiment for implantable devices and the corresponding method for assembly.

This object is achieved for an implantable device according to the preamble of Claim 1 by the features of the characterizing part of Claim 1. The assembly method according to the present invention is the subject matter of Claim 11. Further details of various embodiments and advantages of the present invention are the subject matter of the particular subclaims, which refer back to the independent claims.

To produce implantable devices, it is suggested that the housing be assembled from prefinished plastic parts. In particular, biocompatible materials, such as polyurethane or epoxide resin, are suitable as plastic here. In the simplest case, the housing may comprise two half shells, but it may also be formed by multiple plastic parts for special shapes. An essential advantage is that housing parts made of plastic may be produced very cost-effectively—e.g., in the injection molding method. In addition, plastic parts may be connected to one another very easily and thus cost-effectively using known joining technologies. Preferably, gluing, ultrasonic welding, or thermal joining are used here. A housing according to the present invention may be coated with a special plastic after the assembly, for example, to achieve the required tightness. In the long-term test, parylene has proven itself as a seal layer, for example.

The production of plastic housing parts permits manifold special shapes and embodiments without particular additional costs. Therefore, product-specific housing requirements for various types and embodiment of implantable devices may be taken into consideration. For example, depressions may be provided in the housing parts, which are used for receiving assemblies (power source, electronic modules, etc.) during assembly. These depressions are preferably designed in such a way that the assemblies are fixed in the correct position. Correspondingly, ribs, frames, etc. may be provided which make it easier to position the various components upon insertion, as well as self-centering elements, such as conical depressions and projections which ensured the exact joining of the housing parts. In special areas, space may remain intentionally for the expansion of the battery due to aging and discharge processes, for example. The battery itself is preferably implemented as a hermetically sealed module.

The wall thicknesses of the plastic housing are not to fall below specific levels for mechanically and electrically insulation requirements. For example, currently used mountable headers for cardiac pacemakers made of polyurethane having wall thicknesses of 0.5 mm are viewed as having long-term stability and sufficient electrical insulation strength.

In a preferred embodiment, at least one plastic housing part is designed in such a way that it contains parts of the electrode supply lines and connection devices—embedded in the plastic material. These include, for example, components such as wiring bands, terminals for screw contacting, or sleeves for later accommodation of spring elements. Such a plastic part may also have the shape of the sockets or terminals required for the later contacting with external electrodes.

The use of plastic parts also offers the possibility of embedding product-specific components in the plastic material, which may then later adopt active or passive functions in the implanted device. These include, for example, antennas for telemetry functions, induction coils, or markers for x-ray detection.

To ensure the electromagnetic compatibility, a Faraday shield may be achieved by coating the interiors of all plastic parts of a housing using a conductive material. This may be performed by known methods, e.g., by vapor deposition using metal. Modern conductive plastics (e.g., polythiophene) may also be used for this purpose.

As an additional protection, electronic circuits may be inserted as metal-encapsulated modules. These modules are preferably embedded using an electrically insulating, curing compound. HF filtering of the electrical supply lines may be integrated in such a module.

For devices in which a reference electrode was formed by the metal housing in the embodiment up to this point, this may be implemented in the plastic embodiment according to the present invention by electrically conductive flat parts inserted specially in the housing surface for this purpose, e.g., in the form of a metal plate. This electrode may already be integrated in the housing part in the production process of the plastic part, i.e., for example, during the injection molding procedure.

Inscriptions for identifying the various components of the implantable device may be performed on the particular parts (battery, modules) themselves. The surface of the mounted plastic housing may be marked by laser inscription, for example, if a reference electrode is embedded in the housing, this metal surface may also be used for this purpose.

### BRIEF DESCRIPTION OF THE DRAWINGS

The present invention is explained in greater detail in the following on the basis of a preferred exemplary embodiment with reference to the figures and the reference signs specified therein. A cardiac pacemaker is selected as an exemplary embodiment for an implantable device having a plastic housing according to the present invention in FIGS. 1 through 3.

FIG. 1 shows an internal view of a plastic shell of a housing for a cardiac pacemaker before insertion of electronic module and battery.

FIG. 2 shows a view of the half shell from FIG. 1 having inserted power source and electronic module.

FIG. 3 shows the join configuration of the two plastic half shells of the cardiac pacemaker from FIG. 1.

### DETAILED DESCRIPTION

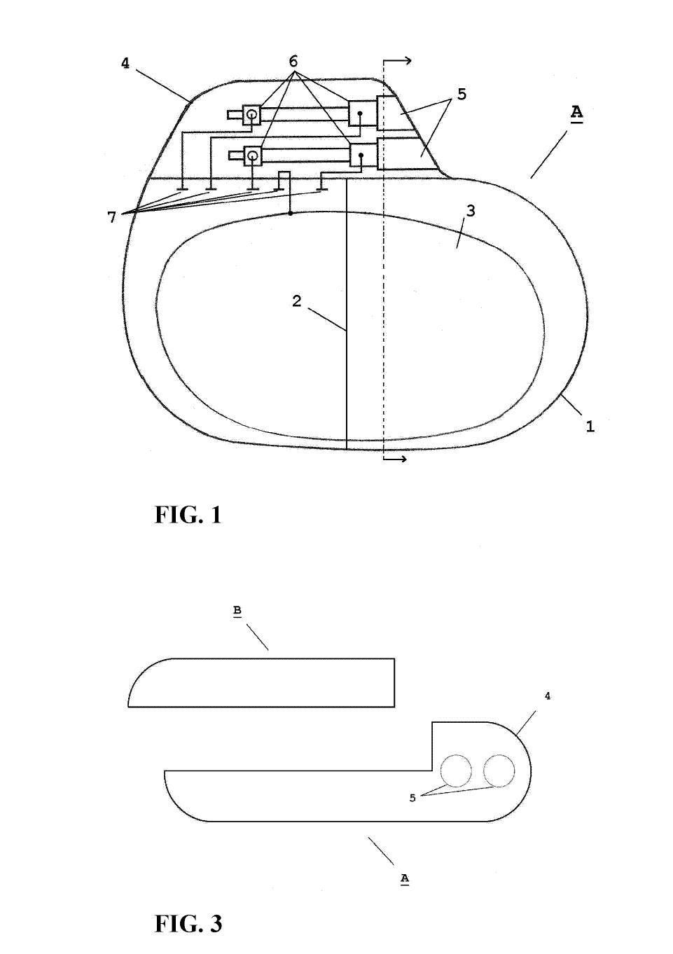

FIG. 1 shows a top view of the interior of a first plastic half shell A; a vertical section through this half shell along the dot-dash line is shown in FIG. 3. The larger, oval area 1 has a concave depression and is provided for receiving the individual components of the cardiac pacemaker. A transverse rib 2 is provided approximately in the middle of the half shell for mechanically fixing the inserted components. A metal plate 3 is embedded in the housing shell for the function of the reference electrode of the cardiac pacemaker. This metal surface produces the contact to the body tissue.

The upper part of the half shell is implemented as a header 4. As may be seen from the section in FIG. 3, this part of the half shell is implemented massively. In this area, the standardized socket openings 5 are located, via which the external electrodes are connected to the cardiac pacemaker. The corresponding units 6 for fixing and contacting are also integrated and embedded in this part of the half shell. A contact array 7 embedded in the half shell produces the connections upon insertion of the electrical module.

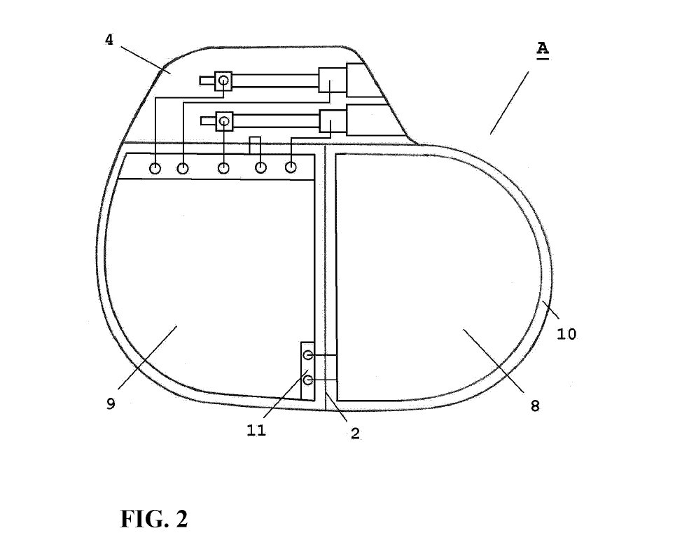

FIG. 2 shows the same half shell having inserted battery 8 and electronic module 9. The transverse rib 2 separates the battery part from the module. The remaining free space 10 around the battery is provided for its expansion upon aging. The electrical connection 11 between battery 8 and module 9 may be implemented here as a contact bridge in the transverse rib 2, for example, or alternatively battery and module may already be electrically connected before insertion, so that only a corresponding through opening is to be provided in the transverse rib.

FIG. 3 shows the two half shells A and B before the assembly. The interior of the half shell B is also shaped concave for the space-saving enclosure of the inserted components. Ribs may also be provided in this half shell to fix battery and module. For precisely fitted assembly of the two half shells during the final assembly, self-centering joining aids may also be provided (not shown).

The present invention offers a cost-effective alternative to titanium housings for implantable devices. In addition, a significant reduction of the component scope is achieved by the use of plastic parts in the housing manufacturing. Thus, depending on the embodiment, various electrical bushings may be dispensed with, as well as welding protection bands (for titanium half shells), internal wiring bands, or insulating films. This has favorable effects on assembly, component supply, and component validation. In addition, the manufacturing sequence for an implantable device is also simplified because complex laser welding technologies are replaced by common joining technologies (e.g., gluing). The time for development is also reduced by these suggestions and the availability is increased by shorter cycle times.

What is claimed is:

1. A housing for an implantable device comprising:
a housing
said housing assembled from prefinished housing parts (A, B); and,
said housing further configured to receive electrical and/or electronic components (8, 9) wherein said prefinished housing parts comprise plastic, in particular biocompatible plastic.

2. The housing according to claim 1 wherein said biocompatible plastic comprises polyurethane or epoxide resin.

3. The housing according to claim 1 wherein said prefinished housing parts (A, B) have depressions in an interior of said housing wherein said depressions are configured to receive said electrical and/or electronic components (8, 9).

4. The housing according to claim 1 wherein said prefinished housing parts have structures configured to mount a device in an interior of said housing wherein said structures comprise ribs (2), frames, and/or self-centering elements.

5. The housing according to claim 1 wherein electrical connections and/or contact bridges (7, 11) from and to said electrical or electronic components (8, 9) are embedded in said prefinished housing parts (A, B).

6. The housing according to claim 1 wherein said prefinished housing parts (A, B) have connection elements, e.g., contact springs, sockets (5, 6), via which external functional elements, such as electrodes, are connected to said implantable device.

7. The housing according to claim 1 further comprising metal electrodes (3) embedded in said prefinished housing parts (A, B) to form a part of an exterior of said housing.

8. The housing according to claim 1 wherein said prefinished housing parts (A, B) are coated on an interior of said housing using a conductive material.

9. The housing according to claim 1 wherein said prefinished housing parts (A, B) are coated on an exterior of said housing using a biologically compatible material,

10. The housing according to claim 9 wherein said biologically compatible material is parylene.

11. The housing according to claim 1 further comprising active functional elements embedded in plastic material of said prefinished housing parts (A, B).

12. The housing according to claim 11 wherein said functional elements comprise at least one antenna or at least one induction coils.

13. The housing according to claim 1 further comprising passive functional elements embedded in plastic material of said prefinished housing parts (A, B).

14. The housing according to claim 13 wherein said passive functional elements comprise markers for x-ray detection.

15. A method for assembling an implantable device comprising:
receiving device components (8, 9) by a housing;
assembling said housing from prefinished plastic parts (A, B); and,
connecting said prefinished plastic parts to one another using a joining technology.

16. The method according to claim 15 wherein said joining technology comprises gluing.

17. The method according to claim 15 wherein said joining technology comprises ultrasonic welding.

18. The method according to claim 15 wherein said joining technology comprises thermal joining.

19. The method according to claim 15 further comprising:
coating an assembled housing with plastic.

20. The method according to claim 19 wherein said plastic is parylene.
